(19) United States
(12) Patent Application Publication
(10) Pub. No.: US 2019/0216871 A1
(43) Pub. Date: Jul. 18, 2019

(54) DIABETIC FOOT CREAM

(71) Applicant: KBR Incorporated, San Marcos, CA (US)

(72) Inventor: Kenneth Rehm, San Marcos, CA (US)

(21) Appl. No.: 16/247,981

(22) Filed: Jan. 15, 2019

Related U.S. Application Data
(60) Provisional application No. 62/618,199, filed on Jan. 17, 2018.

(51) Int. Cl. A61K 36/185; A61K 9/06; A61K 47/44; A61K 47/14; A61K 8/37; A61K 8/42; A61K 8/67; A61K 8/9789; A61Q 19/00; A61P 17/02; A61P 17/18

(52) U.S. Cl. CPC A61K 36/185; A61K 9/06; A61K 47/44; A61K 47/14; A61K 8/37; A61K 8/42; A61P 17/18; A61K 8/676; A61K 8/678; A61K 8/9789; A61Q 19/007; A61P 17/02; A61K 8/671

(57) ABSTRACT

A diabetic foot cream composition for use in treating diabetic foot syndrome is described. The composition includes synergistically effective amounts of a moisturizing agent, aloe vera, a plurality of vitamins, urea, and at least one of a plurality of botanical agents. Advantageously, the vitamins include Vitamin A, Vitamin E, Vitamin C, and Vitamin D. The botanical agents can include arnica, blueberry extract, and pomegranate oil. Methods of ameliorating the symptoms of diabetic foot syndrome are likewise described. The method can include applying a diabetic foot cream to the surface of a foot and massaging the cream into the skin's surface. The treatment area can optionally be cleaned prior to application of the diabetic foot cream. In some aspects, the area is cleaned with a medicated pad.

### BACKGROUND OF THE INVENTION

### Cross-reference to Related Applications

This application claims the benefit of U.S. Provisional Application No. 62/618,199, entitled Diabetic Foot Cream and filed Jan. 17, 2018, the contents of which are incorporated herein by reference in their entirety.

### Field of the Invention

Compositions and methods for treating foot conditions caused or exacerbated by diabetes are provided. More particularly, an all-natural diabetic foot cream is described as well as methods of using said foot cream to ameliorate the common symptoms plaguing the feet of diabetics.

### Description of the Related Technology

It is estimated that at least 415 million people suffer from diabetes mellitus worldwide. According to the American Diabetes Association, the incidence of diabetes has reached epidemic proportions, with approximately 24 million people suffering from diabetes in the U.S. alone. This represents approximately 8% of the adult population. If the current trends continue, 1 in 3 Americans will develop diabetes at some point in their lifetime, and those with diabetes will lose, on average, 10-15 years of life expectancy. The costs associated with diabetes are staggering. In addition to doubling a person's risk of early death, diabetes puts an enormous strain on the U.S. healthcare system, costing as much as $175 billion annually. Indeed, people with diagnosed diabetes have medical expenditures that are over two times higher than medical expenditures for people without diabetes. It is estimated that by the year 2034, the costs for diabetes in the United States will reach $336 billion per year.

One of the complications of diabetes is diabetic foot syndrome. Diabetic foot syndrome includes a combination of conditions often attributed to diabetes such as foot pain caused by diabetes, skin ulcers, neuropathy, and in some instances, gangrene. Foot conditions caused by diabetes is one of the chronic complications of diabetes. Diabetic foot disease can develop through circulatory disorders and in particular, by neuropathy. Additionally, damage to the autonomic nerves can impact the sweat glands in the feet, resulting in increased dryness on the skin of the feet. The skin can become thinner and prone to cracks, fissures, and hyperkeratosis. Moreover, the nails of a diabetic foot are often thicker and drier and can be difficult to trim.

Foot ulcers are another manifestation of diabetic foot conditions and are generally attributable to lower limb vascular stenosis or obstruction, significantly reduced blood supply to the foot, easily associated with severe infections, and wound healing. After ulcers are formed, in severe cases, patients require amputation. Because diabetes is also prone to peripheral nerve lesions, protective foot sensation is diminished or lost and thus, once a patient experiences inflammation and trauma, the patients often cannot perceive the pain and injury in the same fashion as a non-diabetic might perceive them and can result in serious consequences. Thus, proper foot care is essential to reduce the symptoms of diabetic foot.

There remains a need for effective treatment and prevention of diabetic foot syndrome.

### SUMMARY

A new composition and method for treating foot conditions caused or exacerbated by diabetes are disclosed herein. In one aspect, a diabetic foot cream is provided, wherein the cream comprises synergistically effective amounts of shea butter, aloe vera, a vitamin such as Vitamin A, Vitamin E, Vitamin C, Vitamin D, and combinations thereof, urea, blueberry extract, and pomegranate oil. The synergistically effective amount of blueberry extract may be about 0.05% w/w of the composition. The synergistically effective amount of pomegranate oil may be about 0.4% w/w of the composition.

The diabetic foot cream may further include a botanical agent. Suitable botanical agents include, for example, tea tree oil, lavender oil, chamomile, fever few, arnica, calendula, lime tree, lemon myrtle oil, green tea extract, turmeric, yellow birch, and combinations thereof. Optionally, the cream may further include a synergistically effective amount of CoQ10.

In some aspects, the diabetic foot cream may also include a preservative such as pheoxyethenol, caprylyl glycol, potassium sorbate, and hexylene glycol. Advantageously, the foot cream further includes an effective amount of beeswax.

In another aspect, a method of reducing at least one symptom of diabetic foot syndrome is provided. The method includes topically applying to the surface of the foot of an individual in need thereof a diabetic foot cream; and massaging the cream into the surface of the foot; wherein said diabetic foot cream includes a synergistically effective amount of a moisturizing agent, aloe vera, a vitamin, urea, and at least one of a botanical agent. The moisturizing agent may be shea butter. Alternatively, the moisturizing agent may be mink oil, high oleic safflower oil, emu oil, hyaluronic acid, or beeswax. The vitamin may include Vitamin A, Vitamin E, Vitamin C, Vitamin D, and combinations thereof.

In one aspect, the botanical agent is tea tree oil, lavender oil, chamomile, fever few, calendula, lime tree, lemon myrtle oil, green tea extract, turmeric, yellow birch, and combinations thereof. The botanical agent may include arnica, blueberry extract, pomegranate oil, and combinations thereof.

Symptoms of diabetic foot syndrome can include dry skin, cracking, stiffness, fatigue, inflammation, redness, itching, fungal infection, viral infection, bacterial infection, or combinations thereof. In still another aspect, a symptom of diabetic foot syndrome includes neuropathy.

Optionally, the method of treating a symptom of diabetic foot syndrome may further include cleaning the surface of a foot to be treated with a medicated pad prior to topically applying the diabetic foot cream.

In yet another aspect, a composition for treating diabetic foot syndrome is disclosed, wherein the composition includes an effective amount of the following components: blueberry extract; pomegranate extract; Vitamin B; Vitamin D; Vitamin C; and green tea extract.

The composition may be formulated as a lotion or a cream.

### BRIEF DESCRIPTION OF THE DRAWINGS

FIG. 1 is a color photograph of the soles of an individual presenting with diabetic foot syndrome. The foot on the left has been treated with a diabetic foot cream as described herein. The foot on the right has not been treated.

FIG. 2 is a color photograph of the heel and a portion of the sole of the foot of a patient presenting with diabetic foot syndrome.

FIG. 3 is a color photograph of the left foot of the patient's foot from FIG. 2 after being treated with a diabetic foot cream as described herein.

### DETAILED DESCRIPTION

Embodiments of this application relate to a composition for use in treating conditions of the feet which are associated with diabetes. More particularly, embodiments relate to allaying the non-enzymatic glycation process which is causative of various skin and nail conditions of individuals suffering from diabetes. The compositions described herein include natural ingredients which synergistically reduce inflammation, condition skin surface, moisturize, relieve pain, promote circulation, and offer antimicrobial and antioxidant properties to prevent and treat diabetic foot syndrome caused or exacerbated by glycation. Advantageously, the composition is formulated for topical administration as a lotion, cream, salve, ointment, paste, jelly, unction, demulcent, emulsion, or moisturizer.

Formulated specifically as a conditioning treatment for the diabetic and neuropathic foot, the compositions described herein help to lubricate and moisturize the skin, treat dry, cracked, scaly and callused skin, and make the skin more resilient and elastic. The compositions not only add moisture to the skin but also prevent loss of moisture. In certain embodiments, the compositions are applied with a massaging action to the feet, thereby increasing effectiveness and circulation in the foot to help to relieve symptoms of neuropathy.

Turning more particularly to the diabetic foot cream, the composition embodies a unique combination of healing herbs and essential oils which provide a synergistic effect on the healing of the diabetic foot. Effectively, the composition is massaged onto the foot to nourish, condition, hydrate, soothe, and soften skin while delivering key nutrients that help maintain the strength and resilience of the skin and toenails. As will be described in greater detail below, in addition to moisturizing components, the composition comprises an antimicrobial component, humectants which draw water into the outer layer of the skin to decrease dryness, increase flexibility, and resilience; emollients which make the skin softer and more flexible, and an occlusive agent which acts as a physical barrier to epidermal water loss. Preferably, the formulation further includes conditioners. The combination of components work synergistically to promote healthy feet.

The composition further includes an effective amount of vitamins which promote skin softening and healing. An effective amount can include between about 0.05 to about 5% w/w of the formulation. The vitamins can include Vitamin C, Vitamin A, Vitamin E, Vitamin D, and combinations thereof. Vitamin C has antioxidant properties and promotes collagen synthesis. Both dietary and topical Vitamin C have beneficial effects on skin cells, with some studies showing that Vitamin C may help to prevent and treat ultraviolet induced photo damage. Moreover, Vitamin C significantly improves hydration, helping to keep a person's skin looking younger for a longer period of time. It aids the skin's surface in defending itself from external stressors, lessening the effects of exposure to the elements and contaminants. When mixed with other antioxidants, Vitamin C is a powerhouse and can be used to treat hyperpigmentation as well as defend against reactive oxygen species, protecting against oxidative stress. Oxidative stress triggers certain cellular events mediated by transcription factors such as ROS upgrade transcription factor activator protein1 (AP-1) that increases matrix metalloproteinase (MMP) production, leading to collagen breakdown. Oxidative stress induces nuclear transcription factor kappa B (NFkB). This produces a number of mediators that contribute to inflammation and skin aging. ROS has also been linked to the increase in elastin mRNA level in dermal fibroblasts, which may explain the changes in elasticity observed in photo-aged skin.

Vitamin C, in concert with Vitamin E, synergistically limit chronic UV damage by significantly reducing both cell apoptosis and thymine dimer formation. Vitamin C reduces inflammation and promotes wound healing as well as preventing post-inflammatory hyperpigmentation. The composition further includes Vitamin A, which reduces friability and increases blood flow to the surface of the skin. Vitamin A increases the deposition of collagen to promote healing and skin integrity. A powerful skin protectant, Vitamin A moisturizes as it seals the skin and promotes skin healing. Vitamin E, both a nutrient and antioxidant, is highly beneficial to the skin and protects against free radical and environmental damage to the skin. Vitamin E blocks free radicals and boosts collagen production, the connective tissue that keeps skin elastic. Vitamin E oil ameliorates skin inelasticity by promoting skin cell growth and hastening cell regeneration. As a skin protectant and moisturizer, it seals moisture in the skin and promotes healing. Vitamin E provides the skin with necessary moisture as well as antioxidants for intense healing of rough xerotic skin. It restores lost moisture, neutralizes free radical damage, and soothes trauma to the skin. Also a heavy emollient, Vitamin E can act as a cleansing agent. The topical application of Vitamin E can result in the accumulation of Vitamin E not only in cell membranes but also in the extracellular lipid matrix of the stratum corneum, where Vitamin E contributes to antioxidant defenses. The composition can further include an effective amount of Vitamin D, which preserves skin softness, health, and youthful appearance. It locks in moisture and contributes to skin cell growth, repair, and metabolism, optimizing the skin's immune system and helping to destroy free radicals that can cause premature effects of aging. Vitamin D regulates the expression of cathelicidin, an antimicrobial protein that appears to mediate innate immunity in skin by promoting wound healing and tissue repair. Vitamin D can also have a positive effect on dry skin and xerosis. The combination of Vitamins A, E, C, and D cooperate synergistically to promote moisturization, hasten skin healing, reduce inflammation, and increase foot health.

The diabetic foot cream further includes an effective amount of a moisturizing agent. The moisturizing agent can include, without limitation, at least one of the following: shea butter, hyaluronic acid, mink oil, high oleic safflower oil, emu oil, beeswax, or combinations thereof. In one embodiment, the moisturizing agent is shea butter. Shea butter is seed oil and provides the skin with essential fatty acids and nutrients necessary for collagen production. Most seed oils can be divided into two fractions: 1) saponifiable fraction, which is the moisturizing fraction; and 2) nonsaponifiable fraction, which is the healing fraction. Shea butter is particularly well suited as a moisturizing agent in the disclosed diabetic foot cream because of its substantial healing fraction, which includes nutrients, vitamins, and phytonutrients for healing. Shea butter has been associated with improving healing times for skin wounds, ameliorating symptoms of eczema, skin allergies, and other skin conditions. In addition to possessing a high amount of the healing fraction, shea butter is incredibly moisturizing. The concentration of natural vitamins and fatty acids in shea butter makes it highly nourishing and moisturizing for the skin, improving the skin's natural collagen production, and serving as an anti-inflammatory agent in the skin. Hyaluronic acid adds moisture and revitalizes other layers of the skin so they look and feel softer and smoother.

In some embodiments, the moisturizer can include mink oil. Mink oil contains 17% palmitoleic acid, a fatty acid that shares similarities to sebum, the oil our skin produces naturally. It can penetrate deep into the skin to give it moisture and elasticity and is easily absorbed. In addition to being a potent moisturizer, mink oil rejuvenates the skin by providing nutrition in the form of essential fatty acids to keep the skin healthier. Additionally, mink oil acts as an occlusive, sealing the skin and protecting the skin surface. Mink oil can soften the skin, dissolve away dirt and excess oiliness on the surface of the skin, and relieve itching. High oleic safflower oil is yet another moisturizer suitable for use in the diabetic foot cream. In one embodiment, the moisturizing agent is emu oil. Emu oil is a combination of poly and mono unsaturated fats including the omegas 3, 6 and 9. Emu oil softens the skin and increases elasticity and firmness. Emu oil is readily absorbed into the skin due to its smaller particle size. The lineolic acid in emu oil has positive effects by offering protective benefits from scarring and decreasing scar size. In addition to lineolic acid, emu oil is rich in palmitic acid, oleic acid, and antioxidants. Moreover, emu oil has excellent anti-inflammatory properties and can be applied to the body to ease swollen joints and muscular pain. Emu oil can speed up the healing of skin wounds and burns while providing moisture to the skin's surface. Furthermore, emu oil can inhibit pain associated with inflammation. Emu oil's skin benefits are many and include, without limitation, at least the following benefits: soothes body aches and stiffness, decreases scarring from burns of all types, speeds recovery of burns, reduces friction when used for massage therapy, reduces inflammation of muscles and joints, soothes discomfort from wounds, relieves dryness, eases discomfort from neuropathy, relieves itch, softens calloused feet, relieves athlete's foot and other fungal maladies, provides a natural treatment for wounds and incisions, softens dry skin, and diminishes inflammation.

The diabetic foot cream can include an effective amount of beeswax to condition and moisturize the skin. A natural ingredient, beeswax calms and soothes the skin as well as softening the skin surface. Moreover, it can act as a thickening agent for composition to improve the cream's ability to be spread on the skin. Beeswax possesses a number of favorable qualities for inclusion in a diabetic foot cream. It is protective inasmuch as when beeswax is applied to the skin, it forms a protective barrier and traps moisture in the skin and reduces dryness. Additionally, it acts as a humectant to attract water. Additional properties include providing a pleasant fragrance, soothing the skin as an anti-allergenic, and being a good source of Vitamin A.

The composition described herein can further include an effective amount of a botanical agent to treat causes and symptoms of diabetic foot syndrome. Exemplary botanical agents include, without limitation, tea tree oil, lavender oil, chamomile, fever few, arnica, marigold/calendula, lime tree, lemon myrtle oil, pomegranate oil, green tea extract, turmeric, yellow birch, blueberry extract, and combinations thereof. Tea tree oil is an essential oil that can effectively treat tinea infections of the skin and nails, viral infections, as well as staph infections. Also an antiseptic, tea tree oil cleans the surface of the skin and unblocks sebaceous glands, helping to remove dry and dead skin cells. Its antimicrobial properties relieve itching, redness, and burning and aids in controlling the overproduction of skin cells. Lavender oil is similarly a potent anti-inflammatory, antiseptic, and antimicrobial agent. It can be used to speed up the healing process of wounds, cuts, and burns as it improves the formation of scar tissues. Lavender oil inhibits the bacteria that cause infection, helps to regulate some of the over-excretion of sebum, and can reduce the signs of scarring. Moreover, lavender offers pain relief and improves circulation of the blood in the body. Because it lowers blood pressure, it is often used for hypertension, another condition associated with diabetes. Also, lavender oil can treat tired muscles, especially that of the feet and head and neck. Because lavender is a natural anti-inflammatory, it can soothe, soften, and hydrate rough skin of the feet and boost circulation.

Chamomile possesses anti-inflammatory, anti-fungal, antibacterial, and antiseptic properties. It reduces skin irritants by neutralizing free radicals. Fever few is another natural anti-inflammatory herb akin to a weak topical steroid. It cures the redness and swelling of the skin as well as providing a remedy for dry skin. Arnica montana is yet another botanical agent with powerful anti-inflammatory properties. Often used to treat sports injuries or strains from exercising, arnica can be applied topically to reduce swelling and inflammation. Possessing analgesic properties, arnica relieves pain associated with diabetic foot disorder. Arnica is a natural pain relieve that doesn't just mask the pain, it actually stimulates the body's healing processes. It is especially effective at treating pain, swelling, and inflammation.

Marigold or more commonly, calendula, is a natural botanical agent with pain relieving properties. It contains chemicals that speed up wound healing by increasing blood flow to the affected area and promoting the production of collagen proteins. Calendula possesses healing, anti-septic, and anti-inflammatory effects due to its high concentration of flavonoids. Its powerful anti-inflammatory properties are due in part to its high concentration of linoleic acid and can ameliorate the symptoms of a number of skin conditions such as skin ulcerations, eczema, dryness, and inelastic skin. It can improve skin firmness and hydration as well as increasing blood flow and oxygen to wounds and provides antimicrobial effects as well. Lime tree (Citrus aurantifolia oil) acts as an antiseptic, antiviral, astringent, bactericidal, disinfectant, antipyretic, hemostatic, anti-aging, and restorative agent for the skin. It improves circulation through skin penetration and massage by tonight the blood vessels lending a positive effect on the superficial venous and arterial circulation.

Other botanicals include lemon myrtle oil, an astringent which reduces clogged pores, cleanses, and provides anti-inflammatory properties. Rich in phenolic compounds, folate, lutein, and vitamins A and E, lemon myrtle is a potent anti-microbial and antiseptic agent. Advantageously, the composition can include an effective amount of pomegranate oil. An effective amount of pomegranate oil can be between about 0.1 to about 5 w/w. In one embodiment, an effective amount of pomegranate oil is about 0.5% w/w/v. Pomegranate oil is a powerful antioxidant and anti-inflammatory. Active in reducing the effects of glycation in the skin and improving the healing ability of the skin, pomegranate oil increase dermal cell proliferation and collagen synthesis by providing nutritional support for fibroblasts in dermis. Additionally, pomegranate oil inhibits MMP-1 production, promotes healthy thickening of the skin's outer layer, and decreases inflammation. Pomegranate oil contains punicalagins antioxidants and punicic acid, a form of conjugated linoleic acid that is responsible for many health benefits. It can regenerate the dermis by stimulating type I collagen and inhibit protein and collagen breakdown by inhibiting MMP-1 and preventing fibroblast apoptosis cell death. Pomegranate therefore affects different layers of the skin to enhance its strength and healing.

The polyphenols in green tea can neutralize harmful free radicals. Green tea extract acts as an anti-oxidant and anti-bacterial agent as well as an anti-inflammatory. It encourages a natural tone to the skin, helps draw out impurities, and keeps skin hydrated. The diabetic foot cream can include turmeric, a botanical which contains more than 300 antioxidants. Turmeric is a powerful antiseptic and antimicrobial agent and also acts to promote wound healing, soften heels and decrease dry cracked skin on heels, and minimize redness and inflammation. In a preferred embodiment, the diabetic foot cream includes an effective amount of blueberry extract. Preferably, the amount of blueberry extract is between about 0.01 to about 5% w/w/v. In a particularly preferred embodiment, the amount of blueberry extract is about 0.4% w/w/v. Blueberries are rich in antioxidants, which inhibit the oxidization of other molecules. The antioxidants in blueberry extract react with free radicals and prevent them from causing damage. A cup of wild blueberries has up to 13,427 antioxidants, including Vitamin A and C, as well as flavonoids. The phytochemicals and antioxidants found in the fruit help to neutralize the free radicals, which prevents further damage. They are also helpful in improving circulation by strengthening blood vessels and healing broken capillaries.

The diabetic foot cream can optionally include an effective amount of yellow birch, a botanical skin toner and natural pain killer. Yellow birch possesses both antiseptic and disinfectant properties. Because yellow birch contains salicylic acid and methyl salicylate, it acts as an excellent germicide when applied to the surface of the foot. It can reduce pain and inflammation as well as neutralize free radicals.

In certain embodiments, the composition further includes an effective amount of a humectant. Exemplary humectants include, without limitation, lecithin granules, aloe vera powder, urea, and combinations thereof. Lecithin maintains skin moisture by attracting water like a magnet. Lecithin also can lock in the moisture to keep the skin soft and supple by increasing the amount of water that the stratum corneum can hold. Lecithin helps to repair dry, cracked skin by providing maximum hydration and also may play a role in ridding the skin of surface dry skin cells to create a smoother appearance and feel. Advantageously, the composition comprises an effective amount of aloe vera. As used herein, an effective amount of aloe vera includes at least about 0.4% w/w/v of the formulation. Aloe vera replenishes moisture and promotes the protective layer on skin as well as promotes skin firmness. Rich in antioxidants, aloe vera contains a host of nutrients which are beneficial to a diabetic foot. Additionally, aloe vera provides antiseptic, antifungal, antibacterial, and disinfecting properties. Advantageously, aloe vera further soothes and smooths dry skin while enhancing penetration of dry skin.

An effective amount of urea can also be included in the composition. As used herein, an effective amount of urea may include between about 0.05-9.0% w/w/v of the composition. Urea is a humectant which helps the skin maintain a healthy moisture balance, keeping it soft, supple, youthful, and bringing relief to a dry skin. Urea reduces roughness on a skin surface by maintaining skin hydration. When urea is applied to the skin, it penetrates the stratum corneum, where it readily absorbs and retains water, thus increasing the capacity of the skin to hold moisture and rehydrate. In addition to improving skin moisture, urea is associated as having anti-itch properties and provide a natural exfoliant. Urea has been shown to increase the skin permeability of certain skin care ingredients, working as a vehicle for other performance ingredients, encouraging them to penetrate the epidermis easily. Urea can be present in the composition in an amount of between about 0.05-1.0% w/w. In some embodiments, the composition comprises about 0.1% w/w urea.

The composition can also include an emollient and/or emulsifier in an amount of between about 0.5-5 5 w/w. Suitable emollients/emulsifiers include, for example, glyceryl stearate, stearic acid, borax, and combinations thereof. Glyceryl stearate can be a skin conditioning agent which softens and moisturizes skin. It may also act as a lubricant on the skin's surface to give the skin a softer and smoother appearance. Stearic acid likewise protects the skin surface against water loss and may shore up the skin's protective barrier. Borax can be included in the formulation to emulsify the cream. Optionally, the diabetic foot cream as described herein can include an effective amount of an alcohol such as cetearyl alcohol. Cetearyl alcohol is a fatty alcohol which is non-irritating and can be a beneficial conditioner and softener of the skin. The alcohol can cat as an emollient, emulsifier, thickener, or carrying agent for other components of the composition. As a thickening agent and surfactant, cetearyl alcohol helps to optimize the viscosity of the diabetic foot cream. Other suitable alcohols include fatty or wax alcohols (lauryl, stearyl, and cetyl alcohol) as well as ethanol, ethyl alcohol, denatured alcohol, methanol, isopropyl alcohol, SD alcohol, and benzyl alcohol.

In other embodiments, the composition includes an effective amount of COQ10. As used herein, an effective amount of COQ10 includes from between about 0.5 to 10% COQ10 w/w. COQ10 is an antioxidant and skin soothing component which energizes the skin. It supplies the energy needed to repair damaged skin and promote regeneration of healthy skin cells. Moreover, COQ10 neutralizes harmful free radicals, making the skin less prone to damage, and it reduces the breakdown of collagen and elastin such that the quality of skin treated with COQ10 is firm yet flexible and possesses greater resilience. COQ10 energizes the skin. Young skin has an ample amount of COQ10 and so has lots of energy—energy needed to repair damage and ensure that the skin cells are healthy. CoQ10 protects against photo-aging—meaning premature aging due to over exposing the skin to the sun. Additionally, it rejuvenates the skin by stimulating skin cell activity. Active skin cells get rid of toxins more readily and can make better use of nutrients. Furthermore, CoQ10 stimulates collagen production, a protein that decreases as we age.

The composition can further include an effective amount of an exfoliant to decrease keratinization. Exemplary exfoliants include lactic acid. In one embodiment, the exfoliant is alpha hydroxy acid and decreases keratinization. In another embodiment, the exfoliant can be honey, gluconic acid, fruit enzymes from papaya (papain) and pineapple (bromelain), alpha hydroxyl acids, lactic acid, malic acid, tartaric acid, citric acid, mandelic acid, and glycolic acid as well as beta ydroxy acids such as salicylic acid, trichloracetic acid, hyaluronic acid, willow tree bark, wintergreen oil, and sweet birch.

The composition disclosed herein can include a preservative to improve shelf life. Suitable preservatives include a cap 5 preservative such as phenoxyethanol, caprylyl glycol, potassium sorbate, hexylene glycol and combinations thereof.

The composition for treating diabetic foot syndrome is preferably formulated as a cream and can comprise the various active ingredients described above with a sufficient amount of water to achieve the desired viscosity, texture, and spreadability (between about 30-50% water). In some embodiments, the composition includes an effective amount of xantham gum, a natural ingredient used as a thickening agent, texture enhancer, and stabilizer of emulsions. In certain embodiments, the cream comprises a moisturizing agent, a humectant, vitamins, and botanical agents. The moisturizer can be shea butter; the humectant can be aloe vera, urea, or a combination thereof; the vitamins can be Vitamins A, E, C, and D, and the botanical agents can be selected from the group consisting of arnica, blueberry extract, pomegranate oil, green tea, any tea blends, or a combination thereof.

Methods of treating diabetic foot syndrome are likewise provided. The method includes administering to the feet of an individual in need thereof a synergistically effective amount of a composition comprising shea butter, aloe vera, urea, Vitamins A, E, C, and D, blueberry extract, arnica, green tea/tea blends, and pomegranate oil. This combination of ingredients provide a greater than additive effect on promoting healthy feet for diabetics by retarding the process of oxidation, allaying damage associated with non-enzymatic glycation characteristic of diabetics, and reducing inflammation.

The composition is advantageously massaged into the feet of the individual, wherein the massage action helps to stimulate circulation and absorption of the composition. The method may further include the step of cleaning the area to be treated in advance of administration of the diabetic foot cream. Cleaning can be accomplished by washing the foot by any number of conventional methods. In one embodiment, the treatment area can be cleaned with a medicated cleansing pad such as the pad described in co-pending U.S. Provisional patent application No. 62/618,175, filed on Jan. 17, 2018.

### EXAMPLE 1

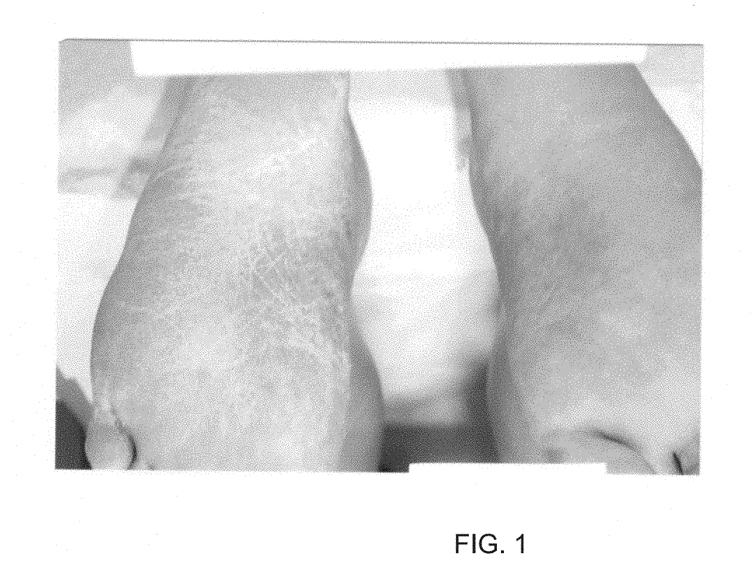

An individual presenting with diabetic foot syndrome was identified. The soles of the individual's feet were dry and cracked. FIG. 1 is a color photograph evidencing the effectiveness of the disclosed diabetic foot cream in reducing symptoms of diabetic foot syndrome. The sole on the left is untreated. Notice the dry and cracked nature of the skin surface. The sole on the right was treated with the disclosed diabetic foot cream. The cream comprising synergistically effective amounts of shea butter, aloe vera, Vitamin B, Vitamin D, Vitamin C, green tea extract, pomegranate oil, and blueberry extract was applied topically to the sole of the foot on the right. The cream was applied daily and massaged into the skin surface. After one month, the signs of diabetic foot syndrome were markedly decreased. The skin surface was smoother, softer, and more supple. The cracks were eliminated and the overall appearance of the foot surface was improved.

### EXAMPLE 2

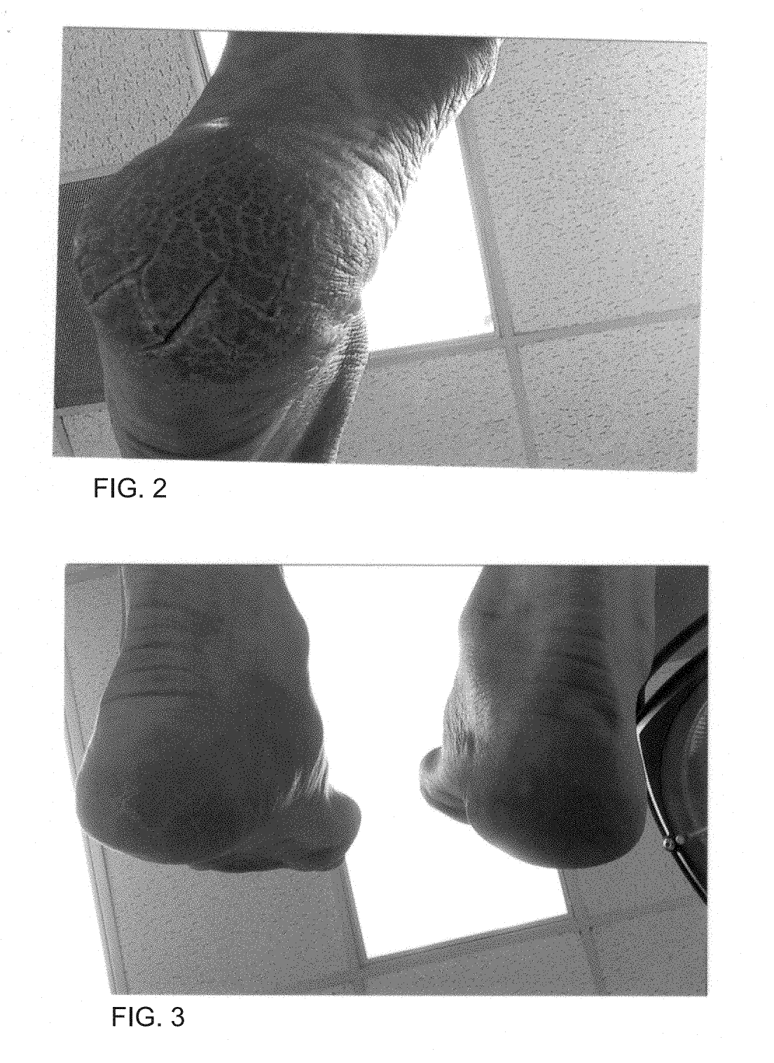

An individual suffering from extremely dry and cracked heels associated with diabetic foot syndrome was identified. As captured in FIG. 2, the patient's heel showed deep, painful cracks and fissures. Circulation was poor and the skin had a dark, yellowed appearance. Note in FIG. 2 the callused, hard skin growth with patchy flakes and cracked and peeling skin. The patient reported that the deep fissures on the heel surface were extremely painful.

The individual was instructed to apply the diabetic foot cream as described herein to the foot surfaces. Application of the diabetic foot cream was performed daily. FIG. 3 is a color photograph showing the feet of the individual photographed in FIG. 2 after five weeks of treatment. Notice the differences in the overall appearance of the foot on the left of FIG. 3 as compared to the foot in FIG. 2. The foot in FIG. 3 shows a marked improvement in overall skin health. The deep fissures in FIG. 2 have been dramatically reduced. The dry, patchy, flaky skin on the heel of FIG. 2 was replaced by smoother, healthier skin. The pigmentation of the skin surface returned to a healthy pink appearance as compared to the dry, discolored appearance in FIG. 2. The patient reported an elimination of pain on standing and walking after treatment with the diabetic foot cream as compared to the pain experienced prior to treatment (and illustrated in FIG. 2). A dramatic improvement in the overall health and appearance of the heel and soles were observed.

### Formulations

One exemplary formulation of the diabetic foot cream as described herein is reflected in Table 1 below:

TABLE 1
Diabetic Foot Cream Formula

| Ingredient | INCI | Percentage |
|---|---|---|
| Water | Water | ~45% |
| Hyaluronic Acid | Sodium Hyaluronate | 1-2.0% |
| Aloe Vera Powder | Aloe barbadensis leaf | 0.1-0.5% |
| Xanthan Gum | Xanthan Gum | 0.1-0.5% |
| Borax | Sodium Borate | .5-2.0% |
| Lactic acid | Lactic acid | .1-1.0% |
| Vitamin C | Ascorbic Acid | .1-.5% |
| Vitamin E (mixed | Tocopherol | 0.1% |
| tocopherols) | | |
| Cap 5 Preservative | Phenoxyethanol, | .1-1.0% |
| | caprylylglycol, potassium | |
| | sorbate, hexylene glycol | |
| COQ10 | COQ10 | 0.025% |
| Vitamin A | Retinyl palmitate | 0.02% |
| Shea Butter | Butyrospermum parkii | 4.0% |
| High Oleic Safflower Oil | Carthamus tinctorius | 5-15% |
| Emu Oil | Emu Oil | 3.0% |
| Mink Oil | Mink Oil | 0.5% |
| Glyceryl stearate | Glycerol stearate | 2.0-3.0% |
| Cetearyl alcohol | Cetearyl alcohol | 1.0-2.0% |
| Stearic acid | Stearic acid | 0.5-1.5% |
| Beeswax | Cera alba | 1-10% |
| Lecithin granules | Lecithin | 0.2% |
| Pomegranate oil | Punica granatum | 1-5% |
| Tea Tree Oil | Malaleuca alternifolia | .1-1.0% |
| Lavender Oil | Lavandula angustifolia | .1-1.0% |
| Lemon Myrtle Oil | Bachousia citriodora | .05-.2% |
| Chamomile | Matricaria recutita flower | 0.8% |
| Fever Few | Chrysanthemum parthenium | 0.4% |
| | oil | |
| Arnica | Arnica montana | 0.8% |
| Marigold | Helianthus anuus, Calendula | 0.4% |
| | officinalis | |
| Blue bottle | Citrus aurantifolia oil | 0.4% |
| Blue berry | Hypericum perforatum | 0.4% |
| Pomegranate | Punica granatum | 0.4% |
| Green Tea Extract | Camellia sinensis | 0.4% |
| Turmeric | Curcuma longa | 0.4% |
| Yellow Birch | Betula alleghaniensis | 0.4% |

In another aspect, the formulation of the diabetic foot cream includes the components set forth below in Table 2:

TABLE 2
Diabetic Foot Cream Formulation #2

| Ingredient | INCI | Percentage |
|---|---|---|
| Water | Water | 58.1% |
| Hyaluronic Acid | Sodium Hyaluronate | 2.0% |
| Aloe Vera Powder | Aloe barbadensis leaf | 0.168% |
| Xanthan Gum | Xanthan Gum | 0.236% |
| Borax | Sodium Borate | .65% |
| Lactic acid | Lactic acid | .6% |
| Vitamin C | Ascorbic Acid | .1-.5% |
| Vitamin E (mixed | Tocopherol | 0.1% |
| tocopherols) | | |
| Cap 5 Preservative | Phenoxyethanol, | .75% |
| | caprylylglycol, potassium | |
| | sorbate, hexylene glycol | |
| COQ10 | COQ10 | 0.025% |
| Vitamin A | Retinyl palmitate | 0.1% |
| Shea Butter | Butyrospermum parkii | 4.0% |
| High Oleic Safflower Oil | Carthamus tinctorius | 9.5% |
| Emu Oil | Emu Oil | 1.0% |
| Mink Oil | Mink Oil | 0.5% |
| Glyceryl stearate | Glycerol stearate | 2.88% |
| Cetearyl alcohol | Cetearyl alcohol | 1.62% |
| Stearic acid | Stearic acid | 1.0% |
| Beeswax | Cera alba | 3% |
| Lecithin granules | Lecithin | 0.2% |
| Pomegranate oil | Punica granatum | 0.5% |
| Tea Tree Oil | Malaleuca alternifolia | .46% |
| Lavender Oil | Lavandula angustifolia | .46% |
| Lemon Myrtle Oil | Bachousia citriodora | .153% |
| Chamomile | Matricaria recutita flower | 0.8% |
| Fever Few | Chrysanthemum parthenium | 0.4% |
| | oil | |
| Arnica | Arnica montana | 0.4% |
| Marigold | Helianthus anuus, Calendula | 0.4% |
| | officinalis | |
| Blue bottle | Citrus aurantifolia oil | 0.4% |
| Blue berry | Hypericum perforatum | 0.4% |
| Pomegranate | Punica granatum | 0.4% |
| Green Tea Extract | Camellia sinensis | 0.4% |
| Turmeric | Curcuma longa | 0.4% |
| Yellow Birch | Betula alleghaniensis | 0.4% |

While certain embodiments are described herein in detail, it is to be understood that this disclosure is illustrative and exemplary, and is merely for the purposes of providing a full and enabling disclosure. The detailed disclosure herein of one or more embodiments is not intended, nor is it to be construed, to limit the scope of the patent protection, for which scope is to be defined by the claims and the equivalents thereof. It is not intended that the scope of patent protection be defined by reading into any claim a limitation found herein that does not explicitly appear in the claim itself.

What is claimed is:

1. A diabetic foot cream, comprising:
a synergistically effective amount of shea butter;
a synergistically effective amount of aloe vera;
a synergistically effective amount of a vitamin selected from the group consisting of Vitamin A, Vitamin E, Vitamin C, Vitamin D, and combinations thereof;
a synergistically effective amount of urea;
a synergistically effective amount of blueberry extract; and
a synergistically effective amount of pomegranate oil.

2. The diabetic foot cream of claim 1, wherein the synergistically effective amount of blueberry extract is about 0.05% w/w of the composition.

3. The diabetic foot cream of claim 1, wherein said synergistically effective amount of pomegranate oil is about 0.4% w/w of the composition.

4. The diabetic foot cream of claim 1, further comprising a botanical agent selected from the group consisting of tea tree oil, lavender oil, chamomile, fever few, arnica, calendula, lime tree, lemon myrtle oil, green tea extract, turmeric, yellow birch, and combinations thereof.

5. The diabetic foot cream of claim 1, further comprising a synergistically effective amount of CoQ10.

6. The diabetic foot cream of claim 1, further comprising a preservative selected from the group consisting of pheoxyethenol, caprylyl glycol, potassium sorbate, and hexylene glycol.

7. The diabetic foot cream of claim 1, further comprising an effective amount of beeswax.

8. A method of reducing at least one symptom of diabetic foot syndrome, comprising:
topically applying to the surface of the foot of an individual in need thereof a diabetic foot cream; and
massaging said cream into the surface of the foot; wherein said diabetic foot cream comprises a synergistically effective amount of a moisturizing agent, aloe vera, a vitamin, urea, and at least one of a botanical agent.

9. The method of claim 8, wherein said moisturizing agent is shea butter.

10. The method of claim 8, wherein said moisturizing agent is selected from the group consisting of mink oil, high oleic safflower oil, emu oil, hyaluronic acid, and beeswax.

11. The method of claim 8, wherein said vitamin is selected from the group consisting of Vitamin A, Vitamin E, Vitamin C, Vitamin D, and combinations thereof.

12. The method of claim 8, wherein said botanical agent is selected from the group consisting of tea tree oil, lavender oil, chamomile, fever few, calendula, lime tree, lemon myrtle oil, green tea extract, turmeric, yellow birch, and combinations thereof.

13. The method of claim 8, wherein said botanical agent is selected from the group consisting of arnica, blueberry extract, pomegranate oil, and combinations thereof.

14. The method of claim 8, wherein said at least one symptom of diabetic foot syndrome is selected from the group consisting of dry skin, cracking, stiffness, fatigue, inflammation, redness, itching, fungal infection, viral infection, bacterial infection, or combinations thereof.

15. The method of claim 8, wherein said at least one symptom is neuropathy.

16. The method of claim 8, further comprising cleaning the surface of said foot with a medicated pad prior to topically applying said diabetic foot cream.

17. A composition for treating diabetic foot syndrome, comprising:
an effective amount of blueberry extract;
an effective amount of pomegranate extract;
an effective amount of Vitamin B;
an effective amount of Vitamin D;
an effective amount of Vitamin C; and
an effective amount of green tea extract.

18. The composition of claim 17, wherein said composition is formulated as a lotion.

19. The composition of claim 17, wherein said composition is formulated as a cream.
